(19) United States
(12) Patent Application Publication
(10) Pub. No.: US 2026/0177539 A1
(43) Pub. Date: Jun. 25, 2026

(54) GENETICALLY ENCODED BRANDING ON THERMOPLASTIC POLYMERS

(71) Applicant: NEOX Public Benefit LLC, New York, NY (US)

(72) Inventors: Neri Oxman, New York, NY (US); Luis Ruben Soenksen Martinez, New York, NY (US); Finn Stirling, Williston Park, NY (US); Kenneth Walker, New York, NY (US)

(21) Appl. No.: 19/340,158

(22) Filed: Sep. 25, 2025

Related U.S. Application Data
(60) Provisional application No. 63/698,740, filed on Sep. 25, 2024.

(51) Int. Cl. G01N 33/44; C12Q 1/6851; C12Q 1/686

(52) U.S. Cl. CPC G01N 33/442; C12Q 1/6851; C12Q 1/686; G01N 2430/60

(57) ABSTRACT

The present disclosure is generally directed to a detectable polymer composition. The polymer composition may include a thermoplastic polymer. Additionally, the composition may include a molecular tag encapsulated within a polymer matrix of the thermoplastic polymer. Also, the molecular tag may include a unique molecular identifier configured to allow detection of the thermoplastic polymer.

### CROSS REFERENCE TO RELATED APPLICATIONS

This application claims the filing benefit of U.S. Provisional Patent Application Ser. No. 63/698,740, filed on Sep. 25, 2024, which is incorporated herein by reference.

### SEQUENCE LISTING

The instant application contains a Sequence Listing which has been submitted electronically in XML format and is hereby incorporated by reference in its entirety. Said XML copy, created on Mar. 5, 2026, is named NPB-109_SL.xml and is 3,187 bytes in size.

### BACKGROUND

As environmental concerns over plastic pollution continue to rise, the development and use of biodegradable plastics have gained significant attention. Biodegradable plastics are designed to break down in natural environments, minimizing long-term ecological impact. However, the degradation process of these materials can be influenced by various environmental factors such as temperature, humidity, microbial activity, and UV exposure, making it essential to monitor their breakdown effectively.

Monitoring the degradation of biodegradable plastics ensures that these materials decompose as intended, leaving a minimal environmental footprint. This process often involves tracking changes in the polymer structure, weight loss, and mechanical properties over time. Moreover, incorporating molecular tags, such as DNA markers, into biodegradable polymers allows for more precise tracking, authentication, and quality control. Such innovative monitoring strategies help validate the performance of biodegradable plastics in real-world applications, ensuring they meet sustainability goals and comply with regulatory standards.

Genetically encoded branding is an emerging technology that holds great promise for creating tamper-proof product identification and improving traceability across various industries. By embedding unique nucleic acid sequences into materials and products, this approach offers unprecedented security and precision for brand authentication, quality control, and counterfeit prevention. However, despite its potential, genetically encoded branding faces several significant hurdles that must be addressed before it can achieve widespread adoption.

One of the key challenges is the complexity and cost associated with integrating nucleic acid tags into manufacturing processes at scale. The synthesis and encapsulation of DNA sequences, along with ensuring their stability during production and throughout the product's life cycle, can be technically demanding and expensive. Additionally, regulatory and ethical concerns around the use of synthetic DNA in consumer goods, especially in sensitive sectors like food and pharmaceuticals, present further obstacles. Moreover, the detection and validation of these DNA tags often require specialized equipment and expertise, which can limit accessibility and create barriers to broad implementation. Addressing these challenges is essential for genetically encoded branding to reach its full potential as a secure and effective solution for product verification and traceability. As such, a need exists in the arts for compositions and methods for improving identification and traceability in plastic-based products using genetically encoded labels.

### SUMMARY

In general, the present disclosure is directed to a detectable polymer composition. The polymer composition may comprise a thermoplastic polymer. Additionally, the composition may include a molecular tag encapsulated within a polymer matrix of the thermoplastic polymer. Also, the molecular tag comprises a unique molecular identifier configured to allow detection of the thermoplastic polymer.

In some instances, the thermoplastic polymer may be biodegradable.

In some instances, the polymer may be selected from the group consisting of a polycarbonate (PC), a polymethyl methacrylate (PMMA), a polyurethane (PU), a polystyrene (PS), a polyamide, a polypropylene (PP), a polyvinyl chloride (PVC), polysulphone, polyvinylacetate (PVA), polyester (PES), a polyethylene terephthalate (PET), a polyethylene (PE), a benzocyclobutene (BCB), a high-density polyethylene (HDPE), a polyvinylidene chloride (PVDC), a low-density polyethylene (LDPE), a high impact polystyrene (HIPS), an acrylonitrile butadiene styrene (ABS), a phenol formaldehyde resin (PF), a melamine formaldehyde (MF), a polyetheretherketone (PEEK), a polyetherimide (PEI), polyimide (PI), a polyether ketone imide, a polylactic acid (PLA), a polytetrafluoroethylene (PTFE), a polymethyl pentene (PMP), a polyether ketone (PEK), a polyether sulfone (PES), a polyphenylene sulfide (PPS), a polytetrafluoroethylene (PTFE), a fluoropolymer, a silicone, an ionomer, a moldable elastomer, an ethylene vinyl alcohol (EVOH), a metallocene polymer and a polyethylene naphthalate material.

In some instances, the polymer may be a polyhydroxyalkanoate (PHA).

In some instances, the PHA may be selected from a group consisting of 3-hydroxypropionate (3HP), 3-hydroxybutyrate (3HB), 3-hydroxyvalerate (3HV), 3-hydroxyhexanoate (3HHx), 3-hydroxyoctanoate (3HO), 3-hydroxydecanoate (3HD), 3-hydroxy-5-phenylvalerate (3HPV), 4-hydroxybutyrate (4HB) and 4-hydroxyvalerate, 4-hydroxybutyrate (4HB), 4-hydroxyvalerate (4HV), or a combination thereof.

In some instances, the PHA may be selected from a group consisting of poly 3-hydroxybutyrate-co-3-hydroxypropionate (P(3HB-co-3HP)), poly 3-hydroxybutyrate-co-4-hydroxybutyrate (P(3HB-co-4HB)), poly 3-hydroxybutyrate-co-4-hydroxyvalerate (P(3HB-co-4HV)), poly 3-hydroxybutyrate-co-3-hydroxyvalerate (P(3HB-co-3HV)), poly 3-hydroxybutyrate-co-3-hydroxyhexanoate (P(3HB-co-3HHx)) and poly 3-hydroxybutyrate-co-5-hydroxyvalerate (P(3HB-co-5HV)).

In some instances, the PHA may be a mixture of a first polymer and a second polymer in a ratio of from about 1:5 to about 5:1.

In some instances, the PHA may be a mixture of poly(3-hydroxybutyrate-co-4-hydroxyvalerate) (P(3HB-co-4HV)) and poly(3-hydroxybutyrate-co-3-hydroxyvalerate) (P(3HB-co-3HV)).

In some instances, the molecular tag may include a nucleic acid.

In some instances, the molecular tag may include DNA.

In some instances, the molecular tag may include at least 10 nucleotide bases.

In some instances, the molecular tag may include a sequence rich in adenine (A) and thymine (T).

Example aspects of the present disclosure provide a method for identifying a polymer. The method may include encapsulating the polymer with a molecular tag comprising a unique molecular identifier. Also, the method may include obtaining a sample comprising the molecular tag following biodegradation of the polymer. Additionally, the method may include performing a polymerase chain reaction (PCR) to amplify the molecular tag. Also, the method may include identifying the polymer based on detection of the molecular tag comprising the unique molecular identifier configured to allow detection of the polymer.

In some instances, the half-life of the molecular tag is greater than the half-life of the polymer.

In some instances, the half-life of the molecular tag is at least 10% greater than the half-life of the polymer.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1 depicts detection of a labeled thermoplastic polymer following degradation of the thermoplastic polymer.

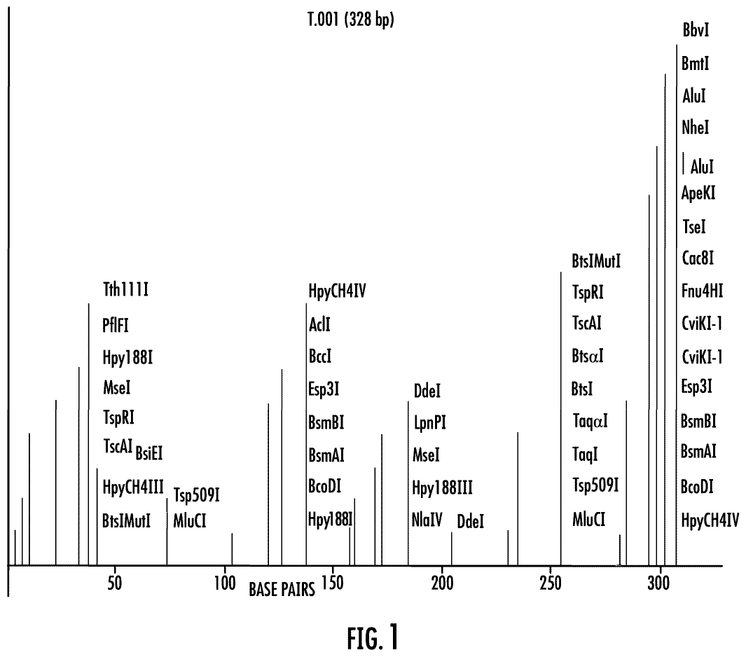

FIG. 2 depicts a flow diagram of an exemplary method for identifying a thermoplastic polymer.

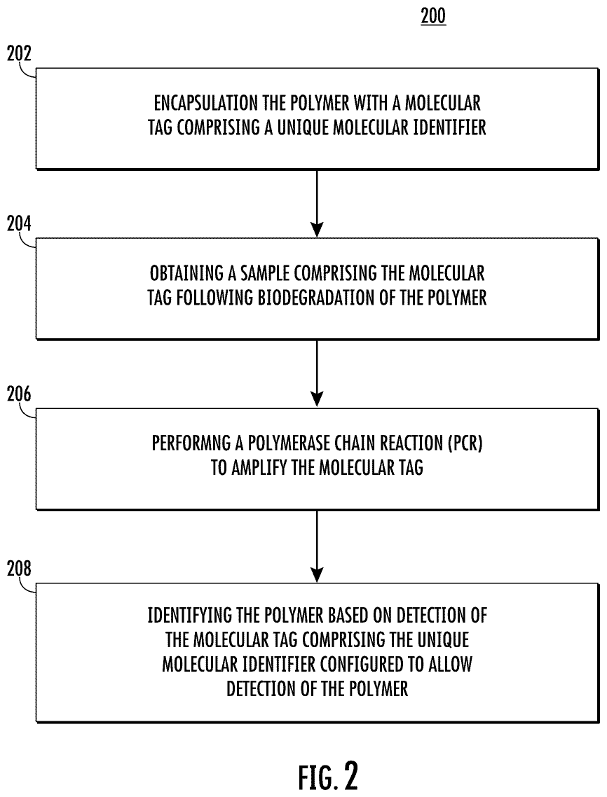

FIG. 3 depicts encapsulation of a synthesized genetic label (302) into PHA.

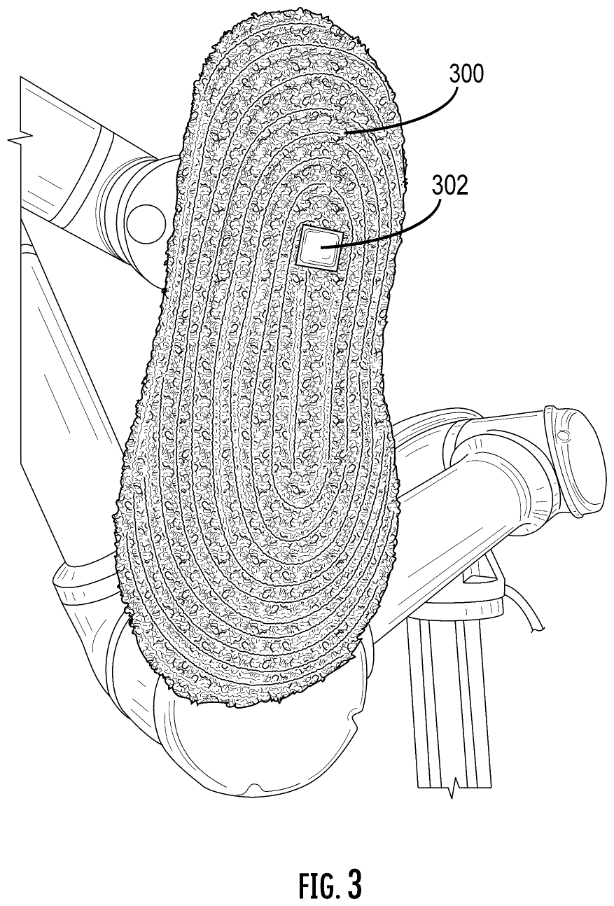

FIG. 4 depicts another angle of encapsulation of a synthesized genetic label (302) into PHA.

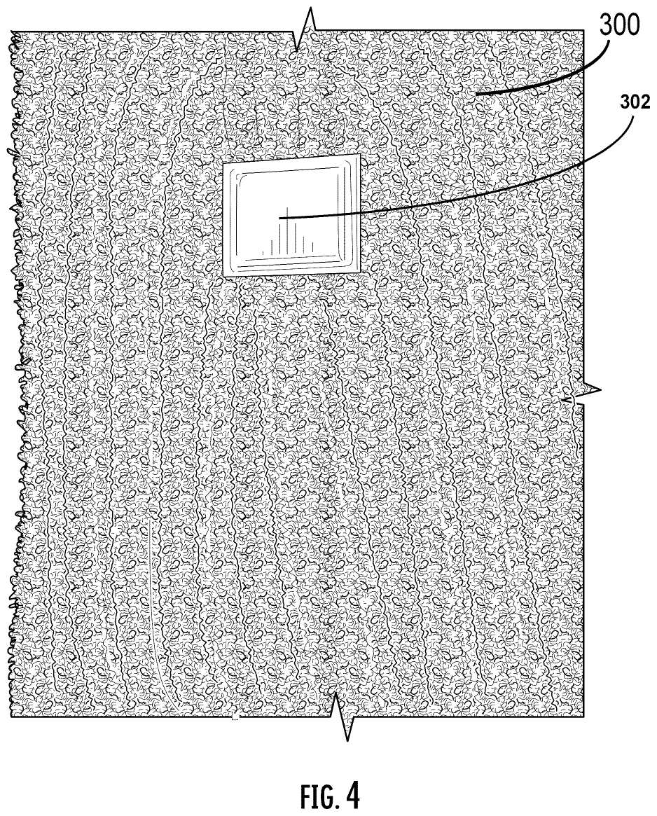

FIG. 5 depicts encapsulation of a synthesized genetic label (302) into PHA.

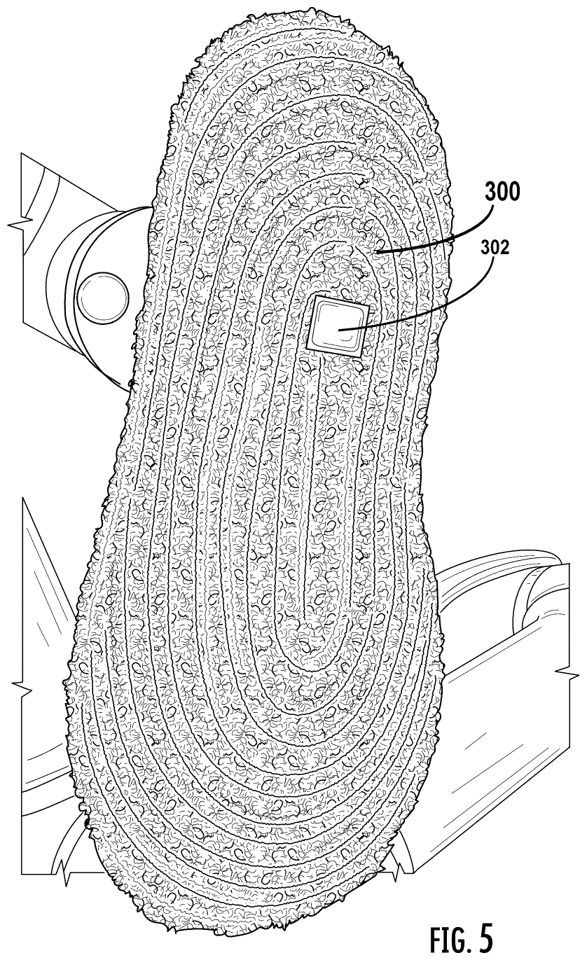

FIG. 6 depicts integration of a synthesized genetic label (302) into PHA.

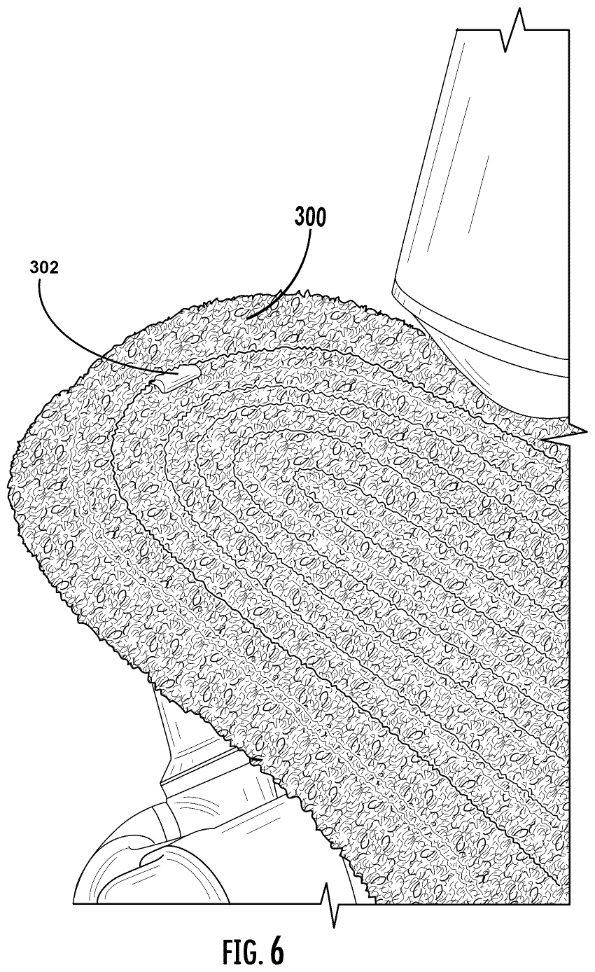

FIG. 7 depicts integration of a synthesized genetic label (302) into PHA from a different angle.

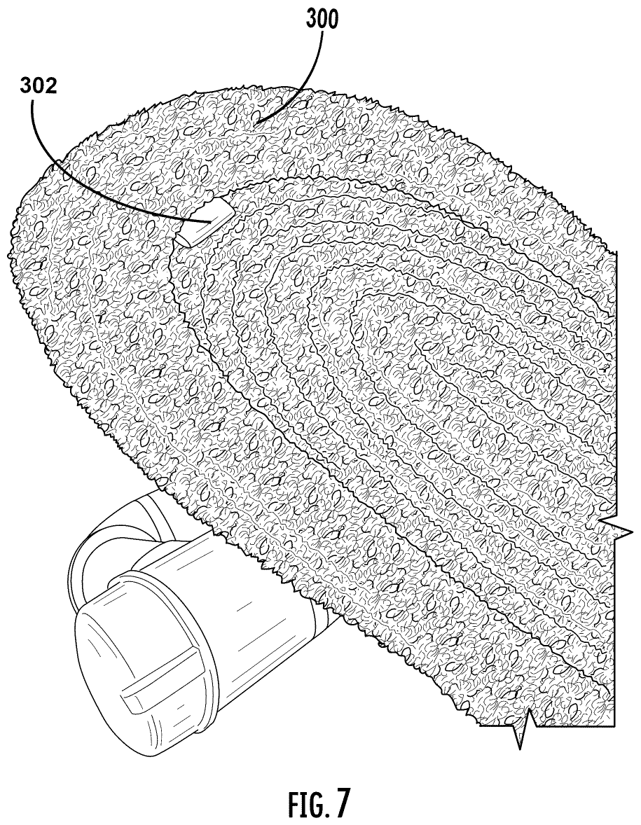

FIG. 8 depicts heat sealing to encapsulate a synthesized genetic label (302) into PHA.

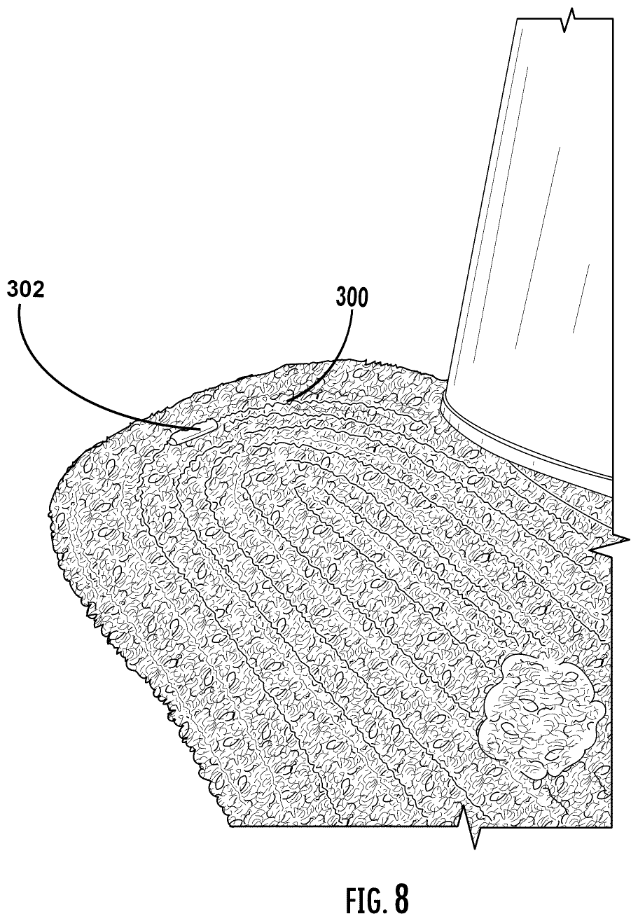

Repeat use of reference characters in the present specification and drawings is intended to represent the same or analogous features or elements of the present invention.

### DETAILED DESCRIPTION

It is to be understood by one of ordinary skill in the art that the present discussion is a description of exemplary embodiments only, and is not intended as limiting the broader aspects of the present disclosure.

In general, the present disclosure is directed to compositions and methods for identifying, detecting, and/or authenticating a biodegradable polymer. Methods disclosed herein may include encapsulating the biodegradable polymer with a genetically encoded label. Beneficially, genetically encoded labels disclosed herein may enable traceability throughout the lifecycle of a plastic product, from production to disposal. This can help manufacturers track the source of materials and ensure compliance with regulatory standards. Additionally, genetically encoded labels may provide a reliable method for identifying and sorting different types of disposed products. This reduces contamination in recycling streams and improves the efficiency of recycling processes.

Genetically encoded labels for thermoplastic polymers represent a cutting-edge approach to improving the branding and traceability of biodegradable plastics. By embedding molecular information directly into products, these labels may offer new value propositions regarding exclusivity, information content, end-of-life traceability, and security. Genetically encoded labels disclosed herein may be a molecular tag. For instance, the genetically encoded label may include, but is not limited to, a genetic sequence or a protein that acts as a label. In some example embodiments, the genetically encoded label may be a protein. In other example embodiments, the genetically encoded label may be a genetic sequence.

In some example embodiments, the genetically encoded label may include a nucleic acid sequence. As used herein, the term “nucleic acid” generally refers to a compound comprising a nucleobase and an acidic moiety, e.g., a nucleoside, nucleotide, polynucleotide, or a combination thereof. A “nucleoside” generally refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” generally refers to a nucleoside including a phosphate group. Modified nucleotides may be synthesized by any useful method, such as, for example, chemically, enzymatically, or recombinantly, to include one or more modified or non-natural nucleosides. Polynucleotides may include a region or regions of linked nucleosides. Such regions may have variable backbone linkages. The linkages may be standard phosphodiester linkages, in which case the polynucleotides would include regions of nucleotides. For instance, polynucleotides may include three or more nucleotides in which adjacent nucleotides are linked to each other via a phosphodiester linkage. The term “nucleic acid” also encompasses RNA as well as single and/or double-stranded DNA. More particularly nucleic acids may be or may include, for example, ribonucleic acids (RNAs), deoxyribonucleic acids (DNAs), threose nucleic acids (TNAs), glycol nucleic acids (GNAs), peptide nucleic acids (PNAs), ethylene nucleic acids (ENA), cyclohexenyl nucleic acids (CeNA) or chimeras or combinations thereof.

Nucleic acids may be naturally occurring, for example, in the context of a genome, a transcript, mRNA, tRNA, rRNA, siRNA, snRNA, plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Nucleic acids can be purified from natural sources, produced using recombinant expression systems and optionally purified, chemically synthesized, etc. The nucleic acids may also include nucleoside analogs, such as analogs having chemically modified bases or sugars, and backbone modifications.

In some example embodiments, the nucleic acid is or may include natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). For instance, the nucleic acid may include natural nucleosides including, but are not limited to, adenosine, thymidine, guanosine, cytidine, or a combination thereof.

Modified nucleotide base pairing may be employed and encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker may be incorporated into nucleic acid tag disclosed herein.

In certain embodiments, the nucleic acid may be a polynucleotide (e.g., RNA polynucleotides, such as mRNA polynucleotides) in which one or more nucleobases has been modified for labeling purposes. For instance, a polynucleotide (e.g., RNA polynucleotide, such as mRNA polynucleotide) may be employed that includes a combination of at least two (e.g., 2, 3, 4 or more) modified nucleobases. For example, suitable modified nucleobases in the polynucleotide may be a modified cytosine, such as 5-methylcytosine, 5-methyl-cytidine (m5C), N4-acetyl-cytidine (ac4C), 5-halo-cytidine (e.g., 5-iodo-cytidine), 5-hydroxymethyl-cytidine (hm5C), 1-methyl-pseudoisocytidine, 2-thio-cytidine (s2C), 2-thio-5-methyl-cytidine, etc.; modified uridine, such as 5-cyano uridine, 4′-thio uridine, pseudouridine (W), N1-methylpseudouridine (m1ψ), N1-ethylpseudouridine, 2-thiouridine (s2U), 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine (mo5U), 5-methoxyuridine, 2′-O-methyl uridine, etc.; modified guanosine, such as α-thio-guanosine, inosine (I), 1-methyl-inosine (m1I), wyosine (imG), methylwyosine (mimG), 7-deaza-guanosine, 7-cyano-7-deaza-guanosine (preQO), 7-aminomethyl-7-deaza-guanosine (preQ1), 7-methyl-guanosine (m7G), 1-methyl-guanosine (m1G), 8-oxo-guanosine, 7-methyl-8-oxo-guanosine, etc.; modified adenine, such as α-thio-adenosine, 7-deaza-adenine, 1-methyl-adenosine (m1A), 2-methyl-adenine (m2A), N6-methyl-adenosine (m6A), 2,6-diaminopurine, etc.; as well as combinations thereof. In some embodiments, the polynucleotide (e.g., RNA polynucleotide, such as mRNA polynucleotide) includes a combination of at least two (e.g., 2, 3, 4 or more) of the aforementioned modified nucleobases.

In some example embodiments, the nucleic acid may include DNA. DNA may be made up of a coding and non-coding region. The coding region of DNA contains sequences of nucleotides (exons) that are transcribed and translated into proteins. In contrast, non-coding regions include introns, which are located within genes but are spliced out during RNA processing, and intergenic regions, which lie between genes. In some example embodiments, the detectable genetically encoded label (e.g., molecular tag) may be a nucleic acid sequence that is inserted into a non-coding region of the DNA. Beneficially, inserting a detectable molecular tag into a non-coding region of the DNA avoids disrupting any functional genes. For instance, the detectable molecular tag may be located within an intron region of the DNA. Alternatively, the detectable molecular tag may be located within an intergenic region of the DNA. The particular molecular tag utilized may be selected based on its compatibility with the particular polymer to be tagged.

In some example embodiments, the molecular tag may be engineered to degrade at a known rate based on specific environmental conditions. For instance, the average half-life of a piece of DNA may be influenced by various factors such as temperature, pH, and environmental conditions. The length of the molecular tag sequence may vary depending on the desired time frame of degradation. For instance, a longer sequence will take longer to degrade compared to a shorter sequence.

In some example embodiments, the molecular tag may include a repetitive sequence of nucleotides that are prone to degradation. For instance, the molecular tag may include a sequence rich in adenine (A) and thymine (T), AT-rich. Beneficially, AT-rich regions are generally more susceptible to environmental degradation.

Advantageously, the molecular tag may have a greater half life compared to the tagged thermoplastic polymer. For instance, the half-life of the molecular tag is at least 10% greater than the half-life of the thermoplastic polymer, such as at least 20% greater, such as at least 50% greater compared to the half-life of the thermoplastic polymer.

Degradation may be monitored utilizing suitable techniques well known in the art. For instance, DNA degradation may be measured utilizing quantitative polymerase chain reaction (qPCR) which measures the efficiency of DNA amplification. Degraded DNA is often less efficient at serving as a template for polymerase enzymes due to fragmentation or chemical modifications. In a qPCR assay, specific DNA sequences are amplified, and the cycle threshold (Ct) value—the point at which amplification is first detected—can be used to assess the extent of DNA degradation. Higher Ct values indicate lower quantities of amplifiable DNA, which may be the result of degradation.

In some example embodiments, the molecular tag may include at least 2 bases, such as at least 10 bases, such as at least 20 bases, such as at least 40 bases, such as at least 100 bases, such as at least 500 bases.

In some example embodiments, the molecular tag may range in length from about 2 bases to about 500 bases, such as from about 20 bases to about 300 bases, such as from about 50 bases to about 200 bases, such as from about 75 bases to about 150 bases, or any range therebetween.

Genetically encoded tags disclosed herein may be utilized to tag any plastics that could benefit from molecular tagging during fabrication, for tracking during use and after disposal. For instance, plastics may include thermoplastic polymers. In some embodiments, the plastic to be molecularly tagged may be selected from the group consisting of a polycarbonate (PC), a polymethyl methacrylate (PMMA), a polyurethane (PU), a polystyrene (PS), a polyamide, a polypropylene (PP), a polyvinyl chloride (PVC), polysulphone, polyvinylacetate (PVA), polyester (PES), a polyethylene terephthalate (PET), a polyethylene (PE), a benzocyclobutene (BCB), a high-density polyethylene (HDPE), a polyvinylidene chloride (PVDC), a low-density polyethylene (LDPE), a high impact polystyrene (HIPS), an acrylonitrile butadiene styrene (ABS), a phenol formaldehyde resin (PF), a melamine formaldehyde (MF), a polyetheretherketone (PEEK), a polyetherimide (PEI), polyimide (PI), a polyether ketone imide, a polylactic acid (PLA), a polytetrafluoroethylene (PTFE), a polymethyl pentene (PMP), a polyether ketone (PEK), a polyether sulfone (PES), a polyphenylene sulfide (PPS), a polytetrafluoroethylene (PTFE), a fluropolymer, a silicone, an ionomer, a moldable elastomer, an ethylene vinyl alcohol (EVOH), a metallocene polymer and a polyethylene naphthalate material.

In some example embodiments, plastics to be molecularly tagged may be polyhydroxyalkanoates (PHAs). PHAs are a family of biodegradable, biocompatible, and biomanufacturable polyesters. PHAs may be produced in nature by bacterial fermentation of sugar or lipids. Also, PHAs may be synthetically produced. More than 100 different monomers may be combined within this family to produce materials. PHAs have properties similar to plastics such as polypropylene due to their high molecular mass.

Examples of monomer units that may be incorporated in polyhydroxyalkanoate polymers include, but are not limited to, 2-hydroxybutyrate, glycolic acid, 3-hydroxybutyrate, 3-hydroxypropionate, 3-hydroxyvalerate, 3-hydroxyhexanoate, 3-hydroxyheptanoate, 3-hydroxyoctanoate, 3-hydroxynonanoate, 3-hydroxydecanoate, 3-hydroxydodecanoate, 4-hydroxybutyrate, 4-hydroxyvalerate, 5-hydroxyvalerate, and 6-hydroxyhexanoate.

Examples of polyhydroxyalkanoate homopolymers may include, but are not limited to, poly 3-hydroxypropionate (P(3HP)), poly 3-hydroxybutyrate (P(3HB)), poly 3-hydroxyvalerate (P(3HV)), poly 3-hydroxyhexanoate (P(3HHx)), poly 3-hydroxyoctanoate (P(3HO)), poly 3-hydroxydecanoate (P(3HD)), poly 3-hydroxy-5-phenylvalerate (P(3HPV)), poly 4-hydroxybutyrate (P(4HB)), poly 4-hydroxybutyrate (P(4HB)), poly 4-hydroxyvalerate (P(4HV)), or a combination thereof.

In some example embodiments, the PHA may be a copolymer (containing two or more different monomer units) in which the different monomers are randomly distributed in the polymer chain. Examples of PHA copolymers may include, but are not limited to, poly 3-hydroxybutyrate-co-3-hydroxypropionate (P(3HB-co-3HP)), poly 3-hydroxybutyrate-co-4-hydroxybutyrate (P(3HB-co-4HB)), poly 3-hydroxybutyrate-co-4-hydroxyvalerate (P(3HB-co-4HV)), poly 3-hydroxybutyrate-co-3-hydroxyvalerate (P(3HB-co-3HV)), poly 3-hydroxybutyrate-co-3-hydroxyhexanoate (P(3HB-co-3HHx)) and poly 3-hydroxybutyrate-co-5-hydroxyvalerate (P(3HB-co-5HV)).

In some example embodiments, the PHA may be a mixture of PHAs. For instance, PHA may include a mixture of a first PHA and a second PHA. The first PHA and the second PHA, for instance, may be present in the mixture at a ratio of from about 1:10 to about 10:1, such as from about 1:5 to about 5:1, such as from about 1:3 to about 3:1, such as from about 1:2 to about 2:1, or any range therebetween. The first and second PHA may be different PHAs.

For instance, the PHA mixture may include poly(3-hydroxybutyrate-co-4-hydroxyvalerate) (P(3HB-co-4HV)) and poly(3-hydroxybutyrate-co-3-hydroxyvalerate) (P(3HB-co-3HV)). The ratio of P(3HB-co-4HV) to P(3HB-co-3HV), for instance, may range from about 1:5 to about 5:1, such as from about 1:2 to about 2:1, such as from about 1:1.5 to about 1.5:1, or any range therebetween. In one example embodiment, the ratio of P(3HB-co-4HV) to P(3HB-co-3HV) may be about 1:1. In another example embodiment, the ratio of P(3HB-co-4HV) to P(3HB-co-3HV) may be about 2.5:1.

In some example embodiments, the thermoplastic polymer may have a molecular weight of from about 100 kilo-Daltons (kDa) to about 1000 kDa, such as from about 200 kDa to about 750 kDa, such as from about 300 kDa to about 500 kDa, or any range therebetween.

Optionally, the thermoplastic polymer may be combined with an excipient to enhance the thermomechanical properties of the thermoplastic polymer. For instance, the thermomechanical properties may include, but not limited to, crystallinity, flexibility, elasticity, modulus, glass transition, thermal stability, and the like. The excipient can be chosen to modify the strength, flexibility, rigidity, durability, resilience, elasticity, weight, wearability, or other properties of the thermoplastic polymer.

In some example embodiments, if desired, the thermoplastic polymer may be combined with an excipient selected from a group consisting of an additional biopolymer, a filler, an additive, a nucleation agent, a chain extender, an antifreeze agent, a colorant/pigment, a lubricant, a plasticizer or a combination thereof.

If desired, the thermoplastic polymer may be combined with an additional biopolymer. For instance, the additional biopolymer may include cellulose or a derivative thereof. By way of example, and without limitation, a cellulose derivative may include an alkyl cellulose (e.g., methyl cellulose, ethyl cellulose, ethyl methyl cellulose), a hydroxy alkyl cellulose (e.g., hydroxyethyl cellulose, hydroxypropyl cellulose, hydroxyethyl methyl cellulose, hydroxypropyl methyl cellulose, ethyl hydroxyethyl cellulose), a carboxy alkyl cellulose (e.g., carboxymethyl cellulose), an organic ester cellulose (e.g., cellulose acetate, cellulose triacetate, cellulose propionate, cellulose acetate propionate, cellulose acetate butyrate), an inorganic acid cellulose (e.g., nitrocellulose, cellulose sulfate), or a combination thereof. In one example embodiment, cellulose may include an alkyl cellulose such as hydroxypropyl cellulose (HPC), methyl cellulose, ethyl cellulose, or combinations thereof.

If desired, the thermoplastic polymer may be combined with a filler material. The filler material may be metallic or non-metallic. For instance, the filler may include, but is not limited to, a metallic powder, metallic fibers, glass fibers, mineral fibers, mineral particles, glass beads, hollow glass beads, glass flakes, polytetrafluoroethylene particles, graphite, boron nitride, titanium oxide, or a combination thereof. In one example embodiment, the filler may be boron nitride. In another example embodiment, the filler may be titanium oxide.

If desired, the metallic filler may be in the form of particles. For instance, the mean particle size may be from about 0.5 microns to about 100 microns, such as from about 0.7 microns to about 75 microns, such as from about 1 micron to about 50 microns, or any range therebetween. In addition, the particles may have a mean particle size such that at least about 90% of the particles pass through a 150 mesh (105 microns), such as at least about 95%, such as at least about 98%, or any range therebetween. Similarly, when metallic flakes may be employed, the metallic flakes may have a thickness of from about 0.4 to about 1.5 microns, such as from about 0.5 to about 1 micron, such as from about 0.6 to 0.9 microns, or any range therebetween. Further, metallic fibers may also have a diameter of from about 1 micron to about 20 microns, such as from about 2 to about 15 microns, such as from about 3 to about 10 microns, or any range therebetween. The metallic fibers may also have an initial length of from about 2 to about 30 mm, such as from about 3 to about 25 mm, such as from about 4 to about 20 mm, or any range therebetween.

The volume average length of the metallic fibers may be from about 5 to about 400 micrometers, such as from about 8 to about 250 micrometers, such as from about 10 to about 200 micrometers, such as from about 12 to about 180 micrometers, or any range therebetween. The metallic fibers may also have a relatively high aspect ratio (average length divided by nominal diameter) to help improve the thermomechanical properties of the resulting thermoplastic polymer. For example, the fibers may have an aspect ratio of from about 2 to about 50, in some embodiments from about 4 to about 40, and in some embodiments, from about 5 to about 20 are particularly beneficial. The fibers may, for example, have a nominal diameter of about 10 to about 35 micrometers, and in some embodiments, from about 15 to about 30 micrometers.

If desired, the thermoplastic polymer may be combined with non-metallic fillers, such as clay minerals. Clay minerals may be particularly suitable for use as non-metallic fillers in the present invention. For instance, clay minerals may include, but are not limited to, talc (Mg₃Si₄O₁₀(OH)₂), halloysite (Al₂Si₂O₅(OH)₄), kaolinite (Al₂Si₂O₅(OH)₄), illite ((K, H₃O)(Al, Mg, Fe)₂ (Si,Al)₄O₁₀[(OH)₂,(H₂O)]), montmorillonite (Na, Ca)_{0.33}(Al, Mg)₂Si₄O₁₀(OH)₂·nH₂O), vermiculite ((MgFe, Al)₃(Al, Si)₄O₁₀(OH)₂.4H₂O), palygorskite ((Mg, Al)₂Si₄O₁₀(OH).4(H₂O)), pyrophyllite (Al₂Si₄O₁₀(OH)₂), etc., as well as combinations thereof.

In some example embodiments, the molecular tag disclosed herein may be integrated into the thermoplastic polymer during synthesis, polymerization, or compounding processes. For instance, the molecular tag may be integrated into a polymer during its synthesis which involves inserting a unique DNA sequence into the polymer matrix to enable the identification, authentication, or tracking of the polymer. For instance, the molecular tag may be designed to have a unique sequence that will serve as an identifier for the polymer. This DNA sequence can be customized with specific barcodes or reference sequences, ensuring uniqueness and enabling later detection and amplification via polymerase chain reaction (PCR).

In some example embodiments, the DNA tag may be encapsulated into a polymer matrix. Alternatively, the polymer may be synthesized first, and then the molecular tag may be covalently attached to the polymer at specific sites through chemical reactions (e.g., click chemistry, esterification, or amidation). Also, the DNA tag may be integrated via emulsion or bulk polymerization. For instance, the polymer may be synthesized via emulsion polymerization or bulk polymerization, then the molecular tag may be encapsulated into the polymer matrix as the polymerization progresses. As such, the DNA is trapped within these encapsulating structures, which are uniformly dispersed in the polymer, protecting the DNA from environmental factors or chemical reactions during synthesis.

In some example embodiments, the molecular tag may be layered between a polymer matrix during synthesis of the polymer. For instance, co-extrusion may be used to embed molecular tags between layers of the polymer. In this process, the polymer may be extruded in layers, and the molecular tag is incorporated as a thin layer between the polymer matrix. Alternatively, the molecular tag may be integrated into the polymer via solvent casting. For instance, the polymer may be dissolved in a suitable solvent and deposited as a thin film. Between each polymer layer, the encapsulated molecular tag may be introduced as a separate layer. Once the solvent evaporates, the polymer layers solidify, with the molecular tags trapped in between. Multiple layers of polymer and DNA can be applied to achieve the desired thickness and functionality.

In some example embodiments, the molecular tag may be utilized to identify, detect, and/or authenticate a thermoplastic polymer. For instance, the thermoplastic polymer may be identified, detected, and/or authenticated based on the molecular tag attached to it. In some embodiments, the molecular tag may be detected quantitatively without being amplified by PCR. In some embodiments, a single stranded DNA tag labeled with a detection molecule (i.e., fluorophore, biotin, etc.) can be hybridized to a complementary probe attached to a solid support to allow for the specific detection of the “detection molecule” configured to the tag. The nucleic acid DNA tag can also be double stranded, with at least one strand being labeled with a detection molecule. With a double stranded DNA (dsDNA) tag, the nucleic acid tag must be heated sufficiently and then quick cooled to produce single stranded DNA, where at least one of the strands configured with a detection molecule is capable of hybridizing to the complementary DNA probe under appropriate hybridization conditions.

In some example embodiments, the thermoplastic may be authenticated based on amplification of its molecular tag (e.g., through the use of PCR). For instance, a signal may be amplified (e.g., through amplification of a detectable signal, e.g., through use of mechanical, electrical, or chemical approaches). Amplification of a genetically encoded tag or signal may be performed in order to detect a tag or signal. In some embodiments, amplification may be performed enabling the amplification itself to be wholly or at least partially authenticating.

To detect the DNA-tagged polymer, the first step involves extracting the DNA embedded within the polymer matrix. Depending on the nature of the polymer, different methods may be employed to release the DNA. If the polymer is soluble or degradable in a particular solvent (e.g., water or an organic solvent), the polymer can be dissolved, and the DNA can be separated from the polymer material. For polymers that are not easily soluble, enzymatic degradation or mechanical disruption (such as grinding or milling) may be used to break down the polymer and release the DNA tag. Once the DNA is released into solution, a standard DNA extraction kit (e.g., phenol-chloroform extraction or a commercially available DNA isolation kit) can be used to purify the DNA. The DNA extraction step ensures that the tag is free from any inhibitory polymer residues that could interfere with PCR.

Following extraction, the DNA is subjected to PCR to amplify the specific DNA sequence that serves as the unique tag. Primers designed to target the specific sequence of the DNA tag are employed in the PCR reaction. These primers bind to regions flanking the DNA tag, allowing for exponential amplification of the target sequence. The PCR reaction mixture typically includes the extracted DNA, forward and reverse primers specific to the tag sequence, a thermostable DNA polymerase (e.g., Taq polymerase), deoxynucleotide triphosphates (dNTPs), and a reaction buffer optimized for polymerase activity.

For instance, amplification-based assays can be used to measure copy number of a molecular tag. In such amplification-based assays, the nucleic acid sequences act as a template in an amplification reaction (e.g., polymerase chain reaction (PCR)). In a quantitative amplification, the amount of amplification product will be proportional to the amount of template in the original sample. Comparison to appropriate controls provides a measure of the copy number.

In some example embodiments, amplification may be done via a single-species approach using specific primers for a particular tagged, degraded thermoplastic polymer, with PCR or qPCR (quantitative PCR); or with high-throughput and next-generation sequencing (NGS) technologies for massively parallel DNA sequencing at high speed and scalability. See Behjati, Sam, and Patrick S Tarpey. “What is next generation sequencing?” Archives of disease in childhood. Education and practice edition vol. 98,6 (2013): 236-8. doi:10.1136/archdischild-2013-304340. NGS has enabled the multi-species approach of DNA meta-barcoding, which is a mass sequencing technique for the simultaneous molecular identification of multiple thermoplastic polymers in a single complex sample.

Methods of “quantitative” amplification are well-known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). The known nucleic acid sequence for the genes is sufficient to enable one of skill in the art to routinely select primers to amplify any portion of the gene. Fluorogenic quantitative PCR may also be used in the methods of the present invention. In fluorogenic quantitative PCR, quantitation is based on amount of fluorescence signals, e.g., TaqMan and SYBR green.

After the PCR reaction, the amplified DNA products may be detected using gel electrophoresis or other DNA analysis methods such as quantitative PCR (qPCR) or DNA sequencing. In gel electrophoresis, for instance, the PCR products are loaded into an agarose gel matrix and subjected to an electric current, which separates the DNA fragments based on size. A DNA ladder (a standard reference of known DNA fragment sizes) is included for size comparison. The amplified DNA tag is visualized using a DNA stain (e.g., ethidium bromide or a safer alternative such as SYBR Green) under UV light, confirming the presence of the DNA tag in the polymer sample.

In some example embodiments, qPCR can be employed to measure the amount of amplified DNA in real-time, providing information on the concentration of the DNA tag in the original polymer. Alternatively, DNA sequencing can be used to verify the exact sequence of the amplified DNA, ensuring it matches the expected molecular tag and confirming the identity of the polymer.

In some embodiments, the molecular tag may be a protein tag. The activity or level of a protein tag can be detected and/or quantified by detecting or quantifying the expressed polypeptide. The polypeptide can be detected and quantified by any of a number of means well-known to those of skill in the art. Aberrant levels of polypeptide expression of the polypeptides encoded by a nucleic acid and functionally similar homologs thereof, including a fragment or genetic alteration thereof (e.g., in regulatory or promoter regions thereof) are associated with the quantity of the protein tagged thermoplastic polymer. Any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like.

The preceding description is exemplary in nature and is not intended to limit the scope, applicability or configuration of the disclosure in any way. Various changes to the described embodiments may be made in the function and arrangement of the elements described herein without departing from the scope of the disclosure.

Unless defined otherwise, all technical and scientific terms used herein have the same meaning as commonly understood by one of ordinary skill in the art to which this invention is related.

As used in this application and in the claims, the singular forms “a”, “an”, and “the” include the plural forms unless the context clearly dictates otherwise. Additionally, the term “includes” means “comprises”. The methods and compositions of the present disclosure, including components thereof, can comprise, consist of, or consist essentially of the essential elements and limitations of the embodiments described herein, as well as any additional or optional ingredients, components or limitations described herein or otherwise useful in the compositions.

Unless otherwise indicated, all numbers expressing quantities of ingredients, properties such as percentages, and so forth, as used in the specification or claims are to be understood as being modified by the term “about”. Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited.

As used herein, “optional” or “optionally” means that the subsequently described material, event or circumstance may or may not be present or occur, and that the description includes instances where the material, event or circumstance is present or occurs and instances in which it does not. As used herein, “w/w %” and “wt %” mean by weight as relative to another component or a percentage of the total weight in the composition.

The term “about” is intended to mean approximately, in the region of, roughly, or around. When the term “about” is used in conjunction with a numerical range, it modifies that range by extending the boundaries above and below the numerical values set forth. Unless otherwise indicated, it should be understood that the numerical parameters set forth in the following specification and attached claims are approximations. At the very least, and not as an attempt to limit the application of the doctrine of equivalents to the scope of the claims, numerical parameters should be read in light of the number of reported significant digits and the application of ordinary rounding techniques.

Here and throughout the specification and claims, range limitations are combined and interchanged, such ranges are identified and include all the sub-ranges contained therein unless context or language indicates otherwise. For example, all ranges disclosed herein are inclusive of the endpoints, and the endpoints are independently combinable with each other.

This written description uses examples to disclose the present disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

Furthermore, certain aspects of the present disclosure may be better understood according to the following examples, which are intended to be non-limiting and exemplary in nature. Moreover, it will be understood that the compositions described in the examples may be substantially free of any substance not expressly described.

### EXAMPLES

### Example 1

### Designing a Predictable, Genetically Encoded DNA Degradable Label

The average half-life of a piece of DNA can be influenced by various factors such as temperature, pH, and environmental conditions. However, for a simplified model where one wants to calculate the average half-life of a piece of DNA with N nucleotides, one can use an empirical formula based on observed decay rates under specific conditions.

The following approximation may be used to calculate the half-life of a piece of DNA:

$${t}_{\frac{1}{2}} = \frac{k}{N}$$

where:

$${t}_{\frac{1}{2}}$$

is the average half-life of the DNA fragment.
N is the number of nucleotides in the DNA fragment.
k is a constant that depends on the specific environmental conditions, such as temperature, pH, presence of nucleases, etc.

Previous studies estimated that the half-life of DNA fragments in freshwater ranged from a few days to several weeks, depending on the specific conditions. Based on this information, a rough estimate for k in a lake or large body of water might be on the order of tens to hundreds of days.

The value of k can vary significantly depending on the environment and number of nucleotides (N) in the target DNA. For example, at neutral pH and room temperature, some studies suggest that for a 100 nucleotide sequence a rough value for k would fall in the range of 200 to 500 years, though this can be much shorter in more aggressive environments. k for the same 100 nucleotide DNA sequence in a lake or large body of water could be around 3000 days base pairs or 700 days base pairs in forest soil.

It's important to note that this is a highly simplified model and the actual decay process of DNA is complex and influenced by many factors. For precise calculations, experimental data specific to the conditions of interest would be necessary.

Creating a genetically encoded DNA degradation “clock” or “timer” involves designing a sequence that degrades at a predictable rate, allowing you to estimate the time elapsed since the DNA was introduced into the environment. To achieve this, you need to consider the following elements: 1) Predictable Degradation Sequence: Use a sequence that degrades at a known rate under specific environmental conditions; and 2) Location within the DNA: The sequence should be placed in a region of the DNA where its degradation can be easily monitored.

### Design Elements:

a. Choice of Sequence: A common choice for such a timer is a repetitive sequence of nucleotides that are prone to degradation. For instance, using a sequence rich in adenine (A) and thymine (T) could be beneficial since AT-rich regions are generally more susceptible to environmental degradation as presented by Blanco, A (2022) Medical Biochemistry (Second Edition). Pages 131-152 Chapter 6—Nucleic acids
b. Length of the Sequence: The length of the sequence should be chosen based on the desired time frame of degradation. Longer sequences will take longer to degrade completely.

For instance, to design a DNA timer to degrade predictably in a forest soil environment, using the (k) value of 700 days base pairs calculated before. If you want the timer to indicate a period of approximately 7 days:

$$a . {t}_{1 / 2} = k / N$$

$$b . Given ( k = 700 ) days \cdot base pairs and {t}_{1 / 2} = 7 days ,$$

$$c . N = K / {t}_{1 / 2} = 100 base pairs$$

Thus, one would use a sequence of approximately 100 base pairs for this timeframe.

### Sequence Placement:

The sequence should be placed in a non-coding region of the DNA to avoid disrupting any functional genes. A suitable region could be within an intron or an intergenic region. Here is an example sequence:

(SEQ ID NO: 1)
5′-ATATATATATATATATATATATATATATATATATATATATATATAT
ATATATATATATATATATATATATATATATATATATATATATATATATA
TATAT-3′

This sequence consists of 50 repeats of the dinucleotide “AT”, making up 100 base pairs.

### Monitoring Degradation:

To monitor the degradation, you could use quantitative PCR (qPCR) to measure the amount of intact sequence over time. By comparing the amount of remaining sequence at various time points, you can estimate the elapsed time based on the known degradation rate.

Overall, methods disclosed herein leverages the known degradation rates and predictable sequence to create a genetically encoded DNA degradation clock or timer.

### Example 2

The process of incorporating genetically encoded labels into thermoplastic polymers involves the following steps:

Step 1—Designing the Label: The first step is to design a genetic sequence or protein that will act as the label. This sequence is chosen based on its ability to be easily detected, its complexity, and its compatibility with the polymer matrix. As proof of concept, the OXMAN team designed the following label:
Illustrative Label Text: DUST from dust, ASHES to ashes. -OX.OZ0.PHA.SHO.FS1.001 [40.768712, −73.993054]
Illustrative Encoded DNA 1 (328 bases):

(SEQ ID NO: 2)
TCACACTGTATATTTTTTAGTTTAACAATCTCTGACTCGGTCGTACAAT
CTATGTTTGAGTGTAACGAACAATAATTTAGTACATATTTTAGACAATG
TATCGGACAATCATTGAGTCCATCTTTGAGACGCACAAACGTTAGGTTC
AACGCTAGGTTCCAGAAACGCTTAATACATAATACGCTTAGTACATAGG
ACGCTATCTTAGAGATACGCAGAAAGAAAGATACAATTCGAGTAAGAAA
CGCAGTGAGTCAGCAAGTGAGATAGACACGAACAAACGTAGTGAGAGAC
GCAGCTAGCTAGAGAGAAAGTTAGTATTGTACTG

To achieve low complexity, the algorithm can be the following: select text to have as little repeated words or tokens as possible; ensure target text is encoded as utf-8; generate hexadecimal representation of encoded text; and encode into DNA based on a ATCG schema.

Step 2—Synthesis and Integration: The genetic label was synthesized in DNA through the services of Twist Biosciences Inc, using a pTwist Kan High Copy vector. Once the synthesized DNA was received it can be used as is or bacteria can be transformed to produce more of it and integrated into the polymer during the synthesis, polymerization or compounding processes. In the prototype, a 2000 ng of freeze-dried DNA was diluted into 85 μL of nuclease-free water. Then 1 uL drops of this solution was deposited on top of 100 μm, 200 μm and 400 μm film of 50% Poly(3-Hydroxybutyrate) [P3HB] and 50% Poly(3-hydroxybutyrate-co-4-hydroxybutyrate) [P3HB4HB]. The droplet was left to dry and covered to prevent contamination. The amount of DNA per tag is, therefore, approximately 23.529 ng. Once dried a top film of the same thickness was placed on top and heat sealed to encapsulate into an air impermeable construct.

Alternatively, encapsulation can be achieved through techniques such as copolymerization, where the genetic material is covalently bonded to the polymer chains, and also with direct compounding of the DNA with the desired polymer or copolymer.

Step 3—Detection and Reading: Once the labeled polymer is produced and protected with the polymer, specific assays (such as PCR for DNA labels or immunoassays for protein tags) can be used to detect and read the genetic information after polymer biodegradation. This allows for precise identification of the sample.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only and is not intended to limit the invention as further described in such appended claims.

What is claimed:

1. A detectable polymer composition, the composition comprising:
a polymer composition comprising a thermoplastic polymer; and
a molecular tag encapsulated within a polymer matrix of the thermoplastic polymer, wherein the molecular tag comprises a unique molecular identifier configured to allow detection of the thermoplastic polymer.

2. The composition of claim 1, wherein the thermoplastic polymer is biodegradable.

3. The composition of claim 1, wherein the polymer is selected from the group consisting of a polycarbonate (PC), a polymethyl methacrylate (PMMA), a polyurethane (PU), a polystyrene (PS), a polyamide, a polypropylene (PP), a polyvinyl chloride (PVC), polysulphone, polyvinylacetate (PVA), polyester (PES), a polyethylene terephthalate (PET), a polyethylene (PE), a benzocyclobutene (BCB), a high-density polyethylene (HDPE), a polyvinylidene chloride (PVDC), a low-density polyethylene (LDPE), a high impact polystyrene (HIPS), an acrylonitrile butadiene styrene (ABS), a phenol formaldehyde resin (PF), a melamine formaldehyde (MF), a polyetheretherketone (PEEK), a polyetherimide (PEI), polyimide (PI), a polyether ketone imide, a polylactic acid (PLA), a polytetrafluoroethylene (PTFE), a polymethyl pentene (PMP), a polyether ketone (PEK), a polyether sulfone (PES), a polyphenylene sulfide (PPS), a polytetrafluoroethylene (PTFE), a fluropolymer, a silicone, an ionomer, a moldable elastomer, an ethylene vinyl alcohol (EVOH), a metallocene polymer and a polyethylene naphthalate material.

4. The composition of claim 1, wherein the polymer is a polyhydroxyalkanoate (PHA).

5. The composition of claim 4, wherein the PHA is selected from a group consisting of 3-hydroxypropionate (3HP), 3-hydroxybutyrate (3HB), 3-hydroxyvalerate (3HV), 3-hydroxyhexanoate (3HHx), 3-hydroxyoctanoate (3HO), 3-hydroxydecanoate (3HD), 3-hydroxy-5-phenylvalerate (3HPV), 4-hydroxybutyrate (4HB) and 4-hydroxyvalerate, 4-hydroxybutyrate (4HB), 4-hydroxyvalerate (4HV), or a combination thereof.

6. The composition of claim 4, wherein the PHA is selected from a group consisting of poly 3-hydroxybutyrate-co-3-hydroxypropionate (P(3HB-co-3HP)), poly 3-hydroxybutyrate-co-4-hydroxybutyrate (P(3HB-co-4HB)), poly 3-hydroxybutyrate-co-4-hydroxyvalerate (P(3HB-co-4HV)), poly 3-hydroxybutyrate-co-3-hydroxyvalerate (P(3HB-co-3HV)), poly 3-hydroxybutyrate-co-3-hydroxyhexanoate (P(3HB-co-3HHx)) and poly 3-hydroxybutyrate-co-5-hydroxyvalerate (P(3HB-co-5HV)).

7. The composition of claim 5, wherein the PHA is a mixture of a first polymer and a second polymer in a ratio of from about 1:5 to about 5:1.

8. The composition of claim 5, wherein the PHA is a mixture of poly(3-hydroxybutyrate-co-4-hydroxyvalerate) (P(3HB-co-4HV)) and poly(3-hydroxybutyrate-co-3-hydroxyvalerate) (P(3HB-co-3HV)).

9. The composition of claim 1, wherein the molecular tag comprises a nucleic acid.

10. The composition of claim 1, wherein the molecular tag comprises DNA.

11. The composition of claim 1, wherein the molecular tag comprises at least 10 nucleotide bases.

12. The composition of claim 1, wherein the molecular tag comprises a sequence rich in adenine (A) and thymine (T).

13. A method for identifying a polymer, comprising:
encapsulating the polymer with a molecular tag comprising a unique molecular identifier;
obtaining a sample comprising the molecular tag following biodegradation of the polymer;
performing a polymerase chain reaction (PCR) to amplify the molecular tag; and
identifying the polymer based on detection of the molecular tag comprising the unique molecular identifier configured to allow detection of the polymer.

14. The method of claim 13, wherein the half-life of the molecular tag is greater than the half-life of the polymer.

15. The method of claim 14, wherein the half-life of the molecular tag is at least 10% greater than the half-life of the polymer.

16. The method of claim 13, wherein the polymer is a thermoplastic polymer.

17. The method of claim 13, wherein the polymer is selected from the group consisting of a polycarbonate (PC), a polymethyl methacrylate (PMMA), a polyurethane (PU), a polystyrene (PS), a polyamide, a polypropylene (PP), a polyvinyl chloride (PVC), polysulphone, polyvinylacetate (PVA), polyester (PES), a polyethylene terephthalate (PET), a polyethylene (PE), a benzocyclobutene (BCB), a high-density polyethylene (HDPE), a polyvinylidene chloride (PVDC), a low-density polyethylene (LDPE), a high impact polystyrene (HIPS), an acrylonitrile butadiene styrene (ABS), a phenol formaldehyde resin (PF), a melamine formaldehyde (MF), a polyetheretherketone (PEEK), a polyetherimide (PEI), polyimide (PI), a polyether ketone imide, a polylactic acid (PLA), a polytetrafluoroethylene (PTFE), a polymethyl pentene (PMP), a polyether ketone (PEK), a polyether sulfone (PES), a polyphenylene sulfide (PPS), a polytetrafluoroethylene (PTFE), a fluropolymer, a silicone, an ionomer, a moldable elastomer, an ethylene vinyl alcohol (EVOH), a metallocene polymer and a polyethylene naphthalate material.

18. The method of claim 13, wherein the polymer is a polyhydroxyalkanoate (PHA).

19. The method of claim 18, wherein the PHA is selected from a group consisting of 3-hydroxypropionate (3HP), 3-hydroxybutyrate (3HB), 3-hydroxyvalerate (3HV), 3-hydroxyhexanoate (3HHx), 3-hydroxyoctanoate (3HO), 3-hydroxydecanoate (3HD), 3-hydroxy-5-phenylvalerate (3HPV), 4-hydroxybutyrate (4HB) and 4-hydroxyvalerate, 4-hydroxybutyrate (4HB), 4-hydroxyvalerate (4HV), or a combination thereof.

20. The method of claim 18, wherein the PHA is selected from a group consisting of poly 3-hydroxybutyrate-co-3-hydroxypropionate (P(3HB-co-3HP)), poly 3-hydroxybutyrate-co-4-hydroxybutyrate (P(3HB-co-4HB)), poly 3-hydroxybutyrate-co-4-hydroxyvalerate (P(3HB-co-4HV)), poly 3-hydroxybutyrate-co-3-hydroxyvalerate (P(3HB-co-3HV)), poly 3-hydroxybutyrate-co-3-hydroxyhexanoate (P(3HB-co-3HHx)) and poly 3-hydroxybutyrate-co-5-hydroxyvalerate (P(3HB-co-5HV)).

21. The method of claim 13, wherein the PHA is a mixture of a first polymer and a second polymer in a ratio of from about 1:5 to about 5:1.

22. The method of claim 21, wherein the PHA is a mixture of poly(3-hydroxybutyrate-co-4-hydroxyvalerate) (P(3HB-co-4HV)) and poly(3-hydroxybutyrate-co-3-hydroxyvalerate) (P(3HB-co-3HV)).

23. The method of claim 13, wherein the molecular tag comprises a nucleic acid.

24. The method of claim 13, wherein the molecular tag comprises DNA.

25. The method of claim 13, wherein the molecular tag comprises at least 10 nucleotide bases.

26. The method of claim 13, wherein the molecular tag comprises a sequence rich in adenine (A) and thymine (T).


SEQUENCE LISTING (2 sequences)

SEQ ID NO: 1 - DNA, 100 bp, synthetic construct
  source 1..100: mol_type = other DNA
atatatatatatatatatatatatatatatatatatatatatatatatatatatatatatatatatatatatatatatatatatatatatatatatatat

SEQ ID NO: 2 - DNA, 328 bp, synthetic construct
  source 1..328: mol_type = other DNA
tcacactgtatattttttagtttaacaatctctgactcggtcgtacaatctatgtttgagtgtaacgaacaataatttagtacatattttagacaatgtatcggacaatcattgagtccatctttgagacgcacaaacgttaggttcaacgctaggttccagaaacgcttaatacataatacgcttagtacataggacgctatcttagagatacgcagaaagaaagatacaattcgagtaagaaacgcagtgagtcagcaagtgagatagacacgaacaaacgtagtgagagacgcagctagctagagagaaagttagtattgtactg